(19) United States
(12) Patent Application Publication
(10) Pub. No.: US 2013/0267787 A1
(43) Pub. Date: Oct. 10, 2013

(54) ILLUMINATED APPARATUS FOR ELECTROCAUTERY AND DEVICES AND METHOD OF USE

(71) Applicant: INNOSPAN ENTERPRISES, INC., Draper, UT (US)

(72) Inventor: Steven Warnock, Salt Lake City, UT (US)

(73) Assignee: Innospan Enterprises, Inc., Draper, UT (US)

(21) Appl. No.: 13/794,075

(22) Filed: Mar. 11, 2013

Related U.S. Application Data
(60) Provisional application No. 61/620,950, filed on Apr. 5, 2012.

(51) Int. Cl. A61B 18/14; A61B 19/00

(52) U.S. Cl. CPC A61B 18/14; A61B 19/5202
USPC 600/249; 606/49; 606/45

(57) ABSTRACT

According to one configuration, an electrocautery electrode for use in electrocautery or electrosurgery is provided which has a light-emitting component disposed thereon. The electrode is configured to be inserted into or positioned between components of standard electrocautery pencils, making wide distribution easier. According to another configuration, a sleeve is provided with a light-emitting component disposed thereon, the sleeve being sized to fit on any standard electrocautery pencil. The light-emitting component on the electrode or sleeve reduces shadows and allows improved illumination of the treatment sight without necessitating purchase of a new electrocautery device.

### BACKGROUND OF THE INVENTION

1. The Field of the Invention

The present invention relates to surgical tools. More specifically, the present invention relates to an illumination apparatus for use on an electrocautery or electrosurgical device.

2. State of the Art

Surgery generally involves cutting, repairing and/or removing tissue or other materials from a body, whether human or animal. These applications are generally performed by cutting tissue, fusing tissue, or tissue destruction. One of the most ubiquitous procedures to perform these functions in surgery is electrocautery. Many surgeons use electrocautery or electrosurgery instruments because their use is typically cleaner, safer, and more efficient than many of the alternatives.

“Electrocautery” or “electrosurgery” devices as used broadly herein are designed to coagulate and seal blood vessels, or incise, cut or destroy tissue with heat emanating from the device, or they perform the same functions by creating heat in the tissue via electrical current passing out of the device. For convenience, either modality is referred to herein collectively as electrocautery unless specified as being one or the other. Depending on the voltage used or the heat applied in electrocautery, the instrument can have varying effects on the patient's body, such as cutting through soft tissue to access a surgical site, or sealing off blood vessels during surgery to keep the site clean and reduce blood loss. Electrocautery may also be used in ablation or removal of undesirable tissue.

An electrocautery device uses a very high frequency, usually upwards of 100 kHz, to minimize the effect of electrical current on nerves and muscles. The voltage for the electrocautery device is generated by an electrosurgical generator or electrosurgical unit (ESU). Likewise, an ESU can produce a high frequency which is converted into heat by a resistive element or a ferromagnetic material to heat an electrocautery device. The ESU is connected to the instrument, such as an electrocautery pencil, which is held by the surgeon. The electrocautery pencil has a handle portion and an exposed blade portion, or electrode, extending from the end. While the electrode, etc., may be blade shaped, it may also be other shapes as well, Therefore, the active projection of the electrocautery device with which the surgeon “cuts” and causes hemostasis will be discussed generally as an electrode regardless of shape and regardless of whether it functions thermally or electrosurgically.

Most electrocautery pencils are disposable, and the electrode cautery tip is generally interchangeable to allow for adaptation to the specific needs of the surgeon and the procedure.

Despite its advantages and common use, one of the persistent challenges of working with an electrocautery pencil is seeing in the depth of a dark wound. Surgical overhead lights, lighted retractors, or lights worn on the surgeon's head are often used, but these lights are not ideal as they can be cumbersome and a shadow may be cast by the surgeon's hand or by the electrocautery device itself and obscure the field.

Some publications show use of an LED light on the body of the pencil; some have powered the lights through batteries; however, none have fully resolved the intricacies of where to place the light and how to proceed without necessitating entire replacement of an electrocautery pencil.

Thus there is a need for an improved device and method for illumination of the surgical field during electrocautery procedures. The improved device and method should provide a light source very close to the field, thus eliminating shadow problems. Additionally, the device and method should be simple and inexpensive to manufacture, making wide distribution easy and keeping costs down.

### SUMMARY OF THE INVENTION

Described herein is an illumination device for use on an electrocautery pencil.

According to one aspect of the present disclosure, a removable electrocautery electrode is provided with a light-emitting component disposed thereon, with the light-emitting component being electrically connected to the electrode. The electrode and light-emitting component receive power from an external power source connected to the electrocautery pencil. This allows the light-emitting component to operate at any time during surgery when current is flowing to the removable electrode, allowing the light to operate free of the limitations of a battery.

According to another aspect of the present disclosure, the light source is located on the electrocautery electrode, thus eliminating shadowing and greatly improving the field view of the surgeon.

According to another aspect of the present disclosure, the electrocautery electrode provided could be used with any standard electrocautery pencil or electrosurgical generator, making wide distribution simple and less expensive than replacing the entire electrocautery pencil or electrosurgical generator.

According to yet another aspect of the present disclosure, the electrocautery electrode could be adapted to existing proprietary tip designs.

According to another aspect of the present disclosure, the light source may be attached to a mount, the mount having a hole therethrough for a standard electrocautery electrode mounting end to extend through. Thus, a surgeon may slide the mounting end of an electrocautery electrode through the mount with the light source, and then into the electrode receptacle of the electrocautery pencil.

According to another aspect of the present disclosure, the light source may be located on a sleeve configured to be attached to the end of a standard electrocautery pencil. The light source may be powered by a battery, and may be located directly on the sleeve, or connected to the sleeve via flexible positioning wires.

These and other aspects of the present disclosure are realized in an electrocautery electrode with a light source as shown and described in the following figures and related description.

### BRIEF DESCRIPTION OF THE DRAWINGS

Various embodiments of the present disclosure are shown and described in reference to the numbered drawings wherein:

FIG. 1 shows a side view of an electrocautery pencil with a removable electrode in accordance with the principles of the present disclosure;

FIG. 2 shows a top view of the electrocautery pencil with a removable electrode shown in FIG. 1;

FIG. 3 shows a close-up, front view of the electrocautery pencil with a removable electrode shown in FIGS. 1 and 2;

FIG. 4 shows a close-up, perspective view of a removable electrocautery electrode in accordance with the principles of the present disclosure;

FIG. 5A shows a close-up, front view of a configuration of the removable electrocautery electrode in accordance with the principles of the present disclosure;

FIG. 5B shows a close-up, front view of an alternate configuration of the removable electrocautery electrode in accordance with the principles of the present disclosure;

FIG. 5C shows a close-up, front view of yet another alternate configuration the removable electrocautery electrode in accordance with the principles of the present disclosure;

FIG. 5D shows a close-up view of yet another configuration of a removable electrocautery electrode in accordance with the principles of the present disclosure.

FIG. 6 shows a close-up, front view of another configuration of an illumination device for illuminating the surgical field of an electrocautery pencil;

FIG. 7A shows a side, perspective view of a standard electrocautery pencil with a configuration of an illumination device shown positioned to be connected to the electrocautery pencil;

FIG. 7B shows a side, perspective view of the standard electrocautery pencil and configuration of an illumination device shown in FIG. 7A with the illumination device connected to the electrocautery pencil;

FIG. 8A shows a side, perspective view of a standard electrocautery pencil with a configuration of an illumination device shown positioned to be inserted onto the electrocautery pencil;

FIG. 8B shows a side, perspective view of the standard electrocautery pencil and configuration of an illumination device shown in FIG. 8A, with the illumination device inserted over the electrocautery pencil;

FIG. 9A shows a perspective view of an alternate configuration of an illumination device; and

FIG. 9B shows a perspective view of yet another alternate configuration of an illumination device.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity. Similarly, not every embodiment need accomplish all advantages of the present invention.

### DETAILED DESCRIPTION

The following description includes various representative embodiments and specific details in order to provide a thorough understanding of the present disclosure. The skilled artisan will understand, however, that the methods and devices described below can be practiced without employing these specific details, or that they can be used for purposes other than those described herein. Indeed, they can be modified and can be used in conjunction with products and techniques known to those of skill in the art in light of the present disclosure.

Reference in the specification to “one configuration” or “a configuration” means that a particular feature, structure, or characteristic described in connection with the configuration is included in at least one configuration and not that any particular configuration is required to has a particular feature, structure or characteristic described herein. The appearances of the phrase “in one configuration” in various places in the specification are not necessarily all referring to the same configuration, and may not necessarily limit the inclusion of a particular element of the invention to a single configuration, rather the element may be included in other or all configurations discussed herein. Thus it will be appreciated that the claims are not intended to be limited by the representative configurations shown herein. Rather, the various representative configurations are simply provided to help one of ordinary skill in the art to practice the inventive concepts claimed herein.

The invention and accompanying drawings will now be discussed in reference to the numerals provided therein so as to enable one skilled in the art to practice the present invention. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

Furthermore, the described features, structures, or characteristics of configurations of the invention may be combined in any suitable manner in one or more configurations. In the following description, numerous specific details are provided, such as examples of products or manufacturing techniques that may be used, to provide a thorough understanding of configurations of the invention. One skilled in the relevant art will recognize, however, that configurations of the invention may be practiced without one or more of the specific details, or with other methods, components, materials, and so forth. In other instances, well-known structures, materials, or operations are not shown or described in detail to avoid obscuring aspects of the invention.

Prior to discussing particular configurations, it should be understood that the present invention is not limited to any particular structures, process steps, or materials discussed or disclosed herein, but is extended to include equivalents thereof as would be recognized by those of ordinary skill in the relevant art. More specifically, the invention is defined by the terms set forth in the claims. It should also be understood that terminology contained herein is used for the purpose of describing particular aspects of the invention only and is not intended to limit the invention to the aspects or configurations shown unless expressly indicated as such. Likewise, the discussion of any particular aspect of the invention is not to be understood as a requirement that such aspect is required to be present apart from an express inclusion of the aspect in the claims.

It should also be noted that, as used in this specification and the appended claims, singular forms such as “a,” “an,” and “the” may include the plural unless the context clearly dictates otherwise. Thus, for example, reference to “a spring” may include one or more of such springs, and reference to “the layer” may include reference to one or more of such layers.

As used herein, the term “substantially” refers to the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing the nearly all of the length of a lumen would be substantially enclosed, even if the distal end of the structure enclosing the lumen had a slit or channel formed along a portion thereof. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

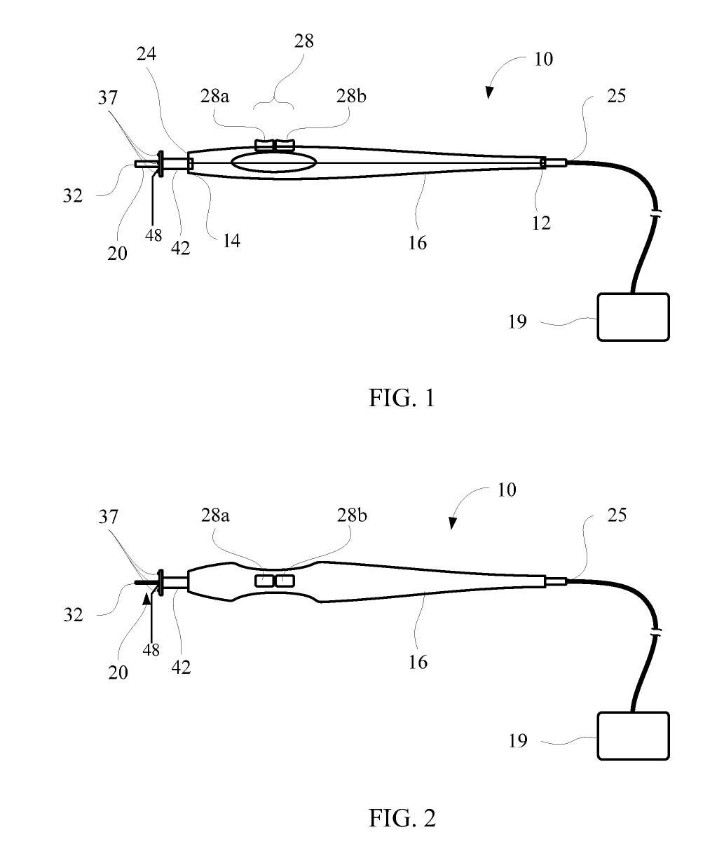

Turning now to FIG. 1, there is shown a side, fragmented view of an electrocautery device, generally indicated at 10. As will be discussed in additional detail below, the electrocautery device 10 may include a body 16 having a proximal end 12 and a distal end 14, a removable electrode 20 according to the present disclosure, and an external power supply 19. As was mentioned previously, the removable electrode 20 may be blade shaped, but it may also be needle shaped or other known configurations.

The body 16 of the electrocautery device 10 may be of the universal type of electrocautery pencil body, with a standard shape which generally forms a handle for grasping by the surgeon. The body 16 may also have other shapes as commonly used with electrocautery devices. The body 16 of the electrocautery device 10 may include a electrode receptacle 24 in the distal end 14 for receiving the removable electrode 20, and a connection 25 on the proximal end 12 for attachment to a cable or line which leads to an external power supply 19. Within the body 16, there may be provided circuitry to electrically connect the blade shaped removable electrode 20 to the external power supply 19 so that power flowing through the handle either heats the removable electrode 20 or causes the removable electrode 20 to transmit current into tissue to thereby cause hemostasis, cutting, etc.

A switch 28 may be disposed on the body 16 and controls the electrical supply to the electrode. The switch 28 may be provided with, for example, two buttons, 28a and 28b. These two buttons, 28a and 28b, may control two different modes for the device, for example cutting and coagulation, respectively. One having skill in the art will appreciate that the switch 28 could also be located remotely from the body 16, such as a foot-controlled switch as is known in the art. The switch 28 may also control additional modes for the device.

The removable electrode 20 (shown in FIG. 1 as being inserted into the electrode receptacle 24 of the electrocautery device 10) may include an electrode tip 32 (or other shaped tip) on the distal end 14, one or more light-emitting components 37, and a connection shaft 42 on the proximal end for engaging the distal end 14 of the body 16. The electrode tip 32, as shown in FIG. 1, may be a standard electrode tip that is flat and sharp. It will be appreciated that numerous other types of electrode tips could be used, such as a needle tip, etc. and may be configurated to act as a thermal element of an electrocautery device or an electrode of an electrosurgical device. Additionally, the electrode tip could be adapted to any proprietary design in the field.

By way of example, the connection shaft 42 of the removable electrode 20 may be round, and may have a diameter of approximately 2.4 mm ( 3/32 inches) in order to be inserted into standard electrocautery pencils. The connection shaft 42 of the removable electrode 20 may also be other various shapes and sizes, configured to be inserted into an electrocautery pencil with a different electrode receptacle 24. The removable electrode 20 may be fabricated from any suitable conductive material, such as, for example, stainless steel, or may be coated with an electrically conductive material. The removable electrode 20 may also be coated in a non-stick material.

Disposed on the removable electrode 20 may be one or more light-emitting components 37. The light-emitting component(s) 37 may be disposed on the removable electrode via a mount 48 (discussed in detail below). The light-emitting component(s) 37 may be electrically connected to the electrode (which receives its power through the electrocautery device 10 from the external power supply 19). Thus, whenever the surgeon presses the switch 28 to send power to the removable electrode 20, the light-emitting component(s) 37 will also receive power and become activated. The light-emitting component(s) 37 may be electrically connected to the electrode through any means suitable and known in the art. According to one configuration, the light-emitting component 37 may be placed in electrical direct contact or direct contact via a conductor contained therein with the conductive removable electrode 20, eliminating the need for any wiring.

Turning now to FIG. 2, there is shown a top view of the electrocautery device 10 shown in FIG. 1. This view shows the ergonomic shape of the body 16 of the electrocautery device 10. One having skill in the art would appreciate that many different designs exist for the shape of electrocautery pencils, and that the present disclosure could be configured to be compatible with any pencil body design.

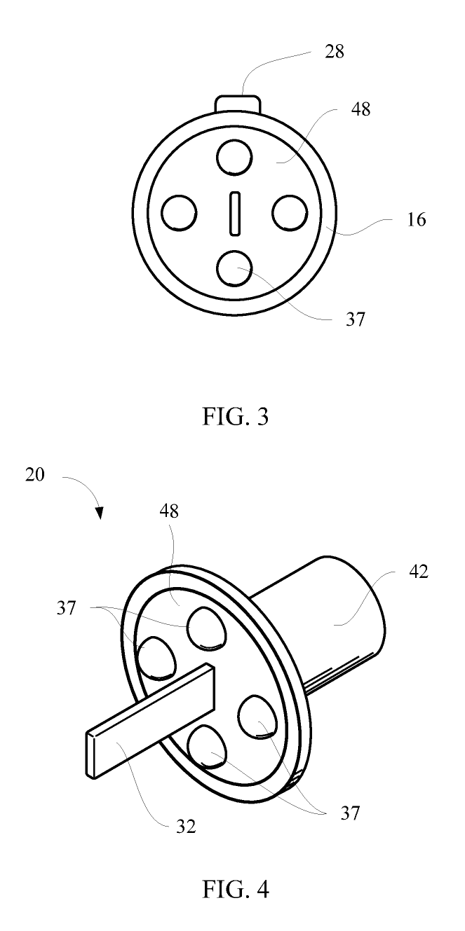

Turning now to FIG. 3, there is shown a front view of the electrocautery device 10 shown in FIGS. 1 and 2. The light-emitting component(s) 37 may be disposed on a mount 48. The mount may be round in shape, so as to be free from abrupt corners. Additionally, the mount 48 may have a diameter smaller than the diameter of the distal end 14 of the body 16 of the electrocautery pencil, so as to not impede movement of the removable electrode 20 in a wound.

Turning now to FIG. 4, there is shown a front perspective view of the removable electrode, generally indicated at 20. The mount 48 may be disposed perpendicular to the length of the removable electrode 20, and the light-emitting component(s) 37 may be disposed on the face of the mount 48 proximal to the electrode tip 32. In FIG. 4, the light-emitting components are shown as four distinct lights, as discussed below. It will be appreciated that different numbers of lights may be used depending on the strength of the lights and the lighting needed in location in which the electrode is being used.

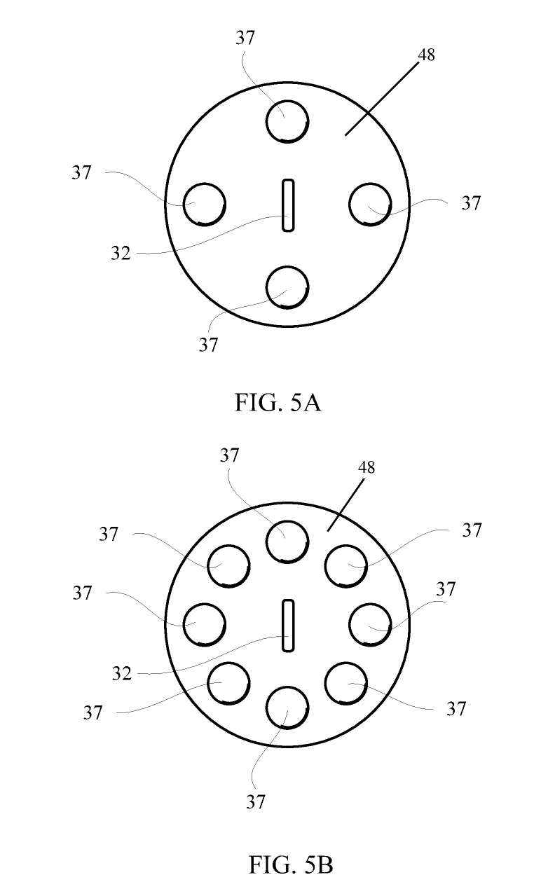

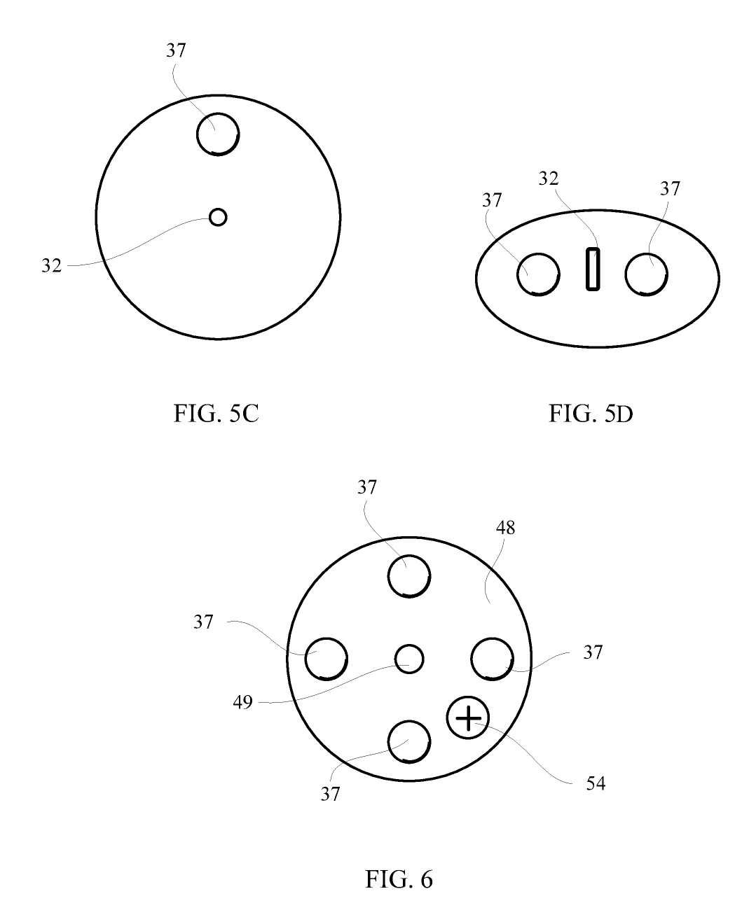

Turning now to FIGS. 5A-D there are shown front views of alternate configurations of the removable electrode 20. FIG. 5A, is a front view of the removable electrode 20 shown in FIG. 4. In this configuration, the light-emitting component(s) 37 are shown as four discrete lights, at approximately 0°, 90°, 180°, and 270°. The light-emitting component may be comprised of light-emitting diodes, such as blue LEDS, white LEDs, yellow LEDs, etc. LEDs may used advantageously because they consume less energy, have a longer lifetime, are available in smaller sizes, and provide faster switching. Other types of lights may also be used, such as fluorescent, incandescent, etc.

FIG. 5B shows a front view of an alternate configuration of a removable electrode 20. In this configuration, the light-emitting component(s) 37 have been increased to eight discrete lights, placed in a circle around the electrode tip 32 and evenly spaced apart. As there is an increase in the light-emitting components, there may be a resulting enhanced illumination of the surgical field. Additionally, the configuration of lights spaced around the electrode tip 32 virtually eliminates any shadow problems.

FIG. 5C shows another alternate embodiment, with a single light-emitting component 37 and a needle-shaped electrode tip 32. One light may be sufficient depending on the surgeon's needs. It is appreciated that there are many various configurations for the light sources, and that the present disclosure encompasses a removable electrode tip with a single light source, multiple light sources, and various configurations of light sources. For example, FIG. 5D shows an oblong mount 48 with light-emitting component(s) 37 disposed on either slide of the electrode tip 32.

According to another possible configuration, the removable electrode 20 may include a mount with the light-emitting component(s) 37 disposed on the mount, but may not include the electrode tip 32. Rather, as shown in FIG. 6, the mount 48 may have a hole 49 disposed therethrough, the hole being sized to allow a standard electrocautery electrode mounting end to fit through the hole 49. The mount 48 may have at least one light-emitting component 37, or multiple light-emitting components. The light-emitting component(s) 37 may receive power from a battery 54, such as a watch battery, disposed on the mount 48. A single battery may be used, or multiple batteries. Alternatively, the light-emitting components 37 may be configured to be electrically attached to the electrocautery electrode 20, as discussed above. Additionally, the mount 48 may have an anchoring mechanism to hold it on the electrocautery electrode 20.

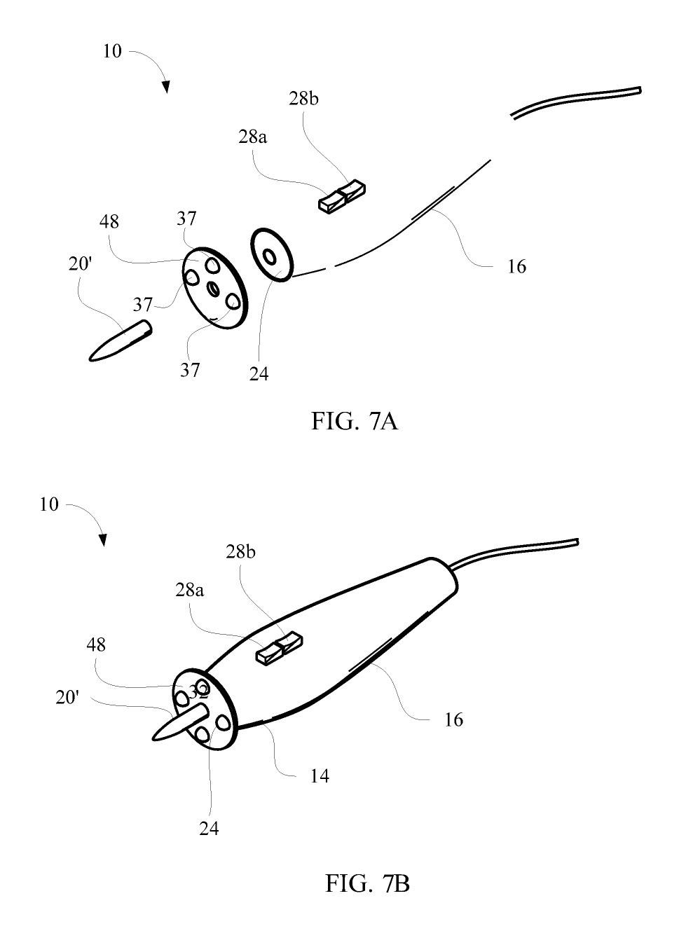

In use, the surgeon may select the mount 48, as shown in FIG. 7A, with the light-emitting component(s) 37; select a standard electrocautery electrode 20′; and select an electrocautery device 10. The mounting end of the standard electrocautery electrode 20′ may be placed through the hole (FIG. 6) of the mount 48, and into the electrode receptacle 24 of the electrocautery device 10. The mount 48 may be held in place by the standard electrocautery electrode 20. FIG. 7B shows a mount 48 connected to an electrocautery device 10 via a standard electrocautery electrode 20′.

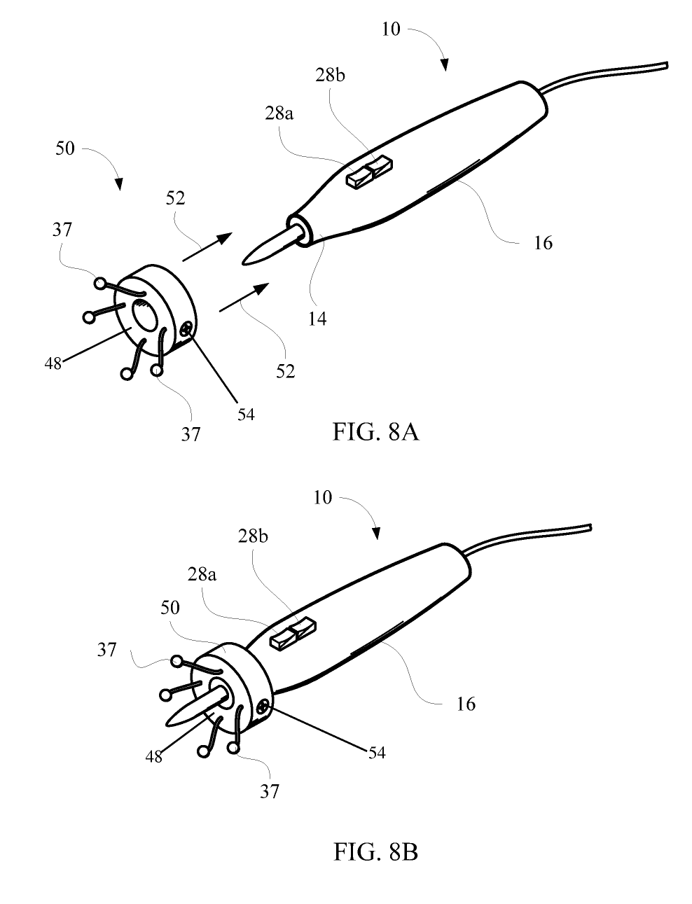

Turning now to FIGS. 8A and 8B, there is shown another possible configuration. According to this configuration, the light-emitting component(s) 37 may be attached to the distal end 14 of the electrocautery device 10 via a sleeve 50. The sleeve 50 may be circular in shape, as the distal end 14 of a standard electrocautery pencil is usually circular in shape. The sleeve 50 may have the light-emitting component(s) 37 attached, and may be inserted over the distal end 14 of the electrocautery device 10 as shown by arrows 52, to fit the sleeve on the electrocautery device 10 as shown in FIG. 8B.

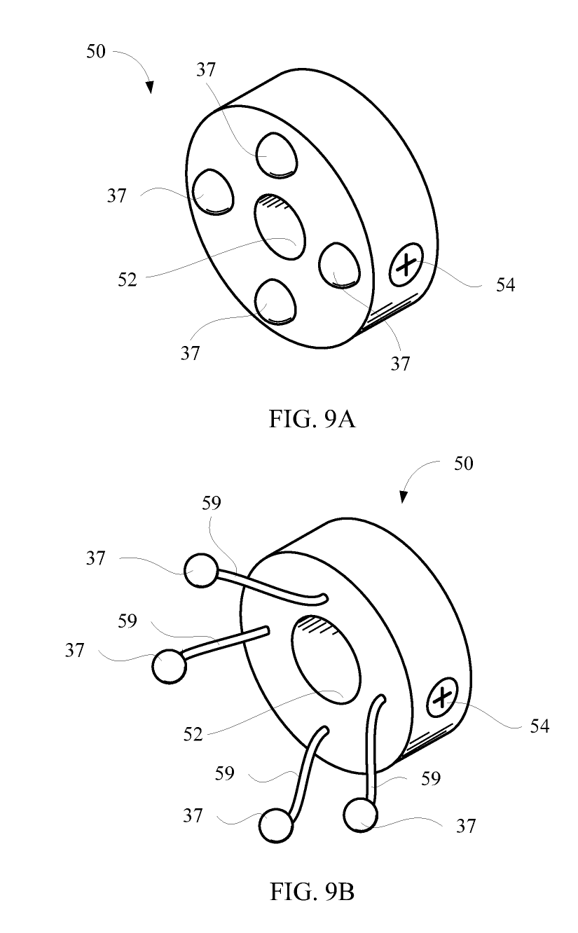

Turning now to FIG. 9A, the sleeve 50 may be circular in shape, with an open interior 52. The open interior 52 may have a diameter similar to the distal end 14 of a standard electrocautery pencil body 16. Thus, according to this configuration, the sleeve 50 with its light-emitting component(s) 37 may be used with nearly any electrocautery pencil.

The sleeve 50 may be fabricated from any suitable material. The sleeve 50 may be fabricated from a material with some amount of elasticity, with the open interior 52 of the sleeve 50 having a diameter slightly smaller than the diameter of the distal end 14 of a standard electrocautery pencil body 16. According to this configuration, the sleeve could be stretched slightly to fit over the distal end 14, positioned at the appropriate location approximate the distal end 14, and then released. When released, the sleeve 50 may fit snugly and not move further without direct manipulation. For example, the sleeve 50 may be formed of silicon rubber, etc.

As shown in FIG. 9A, within the sleeve 50, or on the sleeve 50, may be a power source 54 such as a watch battery. One battery may be used, or multiple batteries may be used. According to the configuration shown in FIG. 9A, four light-emitting components 37 are provided. The light-emitting components 37 may be connected directly to the sleeve 50 as shown in FIG. 9A, or, as shown in FIG. 9B, may be connected to the sleeve 50 via flexible positioning wires 59, fiberoptic line or the like. Flexible positioning wires 59 may allow the surgeon to focus the light-emitting components 37 at the exact position light may be needed.

In use, the surgeon may slip the sleeve 50 over the distal end 14 of the electrocautery device 10, position the flexible positioning wires 59 such that the light-emitting components 37 are in the desired position, and proceed with the surgery. If, during surgery, light is needed at a different position, the surgeon may adjust the flexible positioning wires 59 accordingly.

By placing the light source directly on the electrode, or on the distal end of the electrocautery pencil via a sleeve, the surgeon may have better view of the treatment site and fewer persistent shadow problems. For example, the surgeon may position the light to fall on the tissue immediately where the removable electrode 20 will engage to better see the tissue he or she is about to cut. Additionally, surgeons need not purchase an entirely new electrocautery pencil to realize the advantages described herein. Surgeons need only to buy new electrodes or a new sleeve for use in their current electrocautery pencils, and do not need to replace an entire electrocautery pencil to have additional light. Furthermore, electrical connection of the light-emitting component to the power supply for the electrode allows surgeons to confidently use the light, without worries about battery life.

There is thus disclosed an improved lighting device and method for use in electrocautery pencils. It will be appreciated that numerous changes may be made to the present invention without departing from the scope of the claims.

What is claimed is:

1. An illuminative conductive electrocautery electrode comprising:
a distal end;
a proximal end configured for connection to an electrocautery pencil; and
at least one light emitting component disposed along the electrocautery electrode.

2. The illuminative conductive electrocautery electrode of claim 1, wherein the at least one light emitting component is electrically connected to the electrocautery electrode.

3. The illuminative conductive electrocautery electrode of claim 1, wherein the electrocautery electrode further comprises a mount disposed thereon, and the at least one light emitting component disposed on the mount.

4. The conductive electrocautery electrode of claim 1, wherein the distal end is flattened into a blade-like configuration.

5. The conductive electrocautery electrode of claim 1, wherein the distal end is formed into a needle-like configuration.

6. The conductive electrocautery electrode of claim 1, wherein the at least one light emitting component is comprised of multiple light emitting diodes.

7. The illuminative conductive electrocautery electrode of claim 5, wherein the multiple light emitting diodes are spaced equidistant apart.

8. The illuminative conductive electrocautery electrode of claim 3, wherein the at least one light emitting component disposed on the mount is comprised of multiple light emitting diodes.

9. An electrocautery electrode comprising:
a distal end and a proximal end, and a length extending therebetween;
a post on the proximal end configured for insertion into an electrocautery pencil;
a mount disposed between the distal end and the proximal end, and perpendicular to the length extending therebetween, the mount having a distal face and a proximal face; and
at least one light-emitting component disposed on the distal face of the mount.

10. The electrocautery electrode of claim 9, wherein the at least one light-emitting component and the mount is in electrical connection to the electrocautery electrode.

11. The electrocautery electrode of claim 10, wherein the mount is disposed approximately equidistant between the distal end and the proximal end.

12. The electrocautery electrode of claim 10, wherein the mount is circular in shape.

13. The electrocautery electrode of claim 10, wherein the at least one light-emitting component comprises multiple light emitting diodes.

14. The electrocautery electrode of claim 13, wherein the multiple light emitting diodes are spaced equidistant apart.

15. The electrocautery electrode of claim 10, wherein the distal end is formed into a blade-like configuration.

16. The electrocautery electrode of claim 10, wherein the distal end is formed into a needle-like configuration.

17. An illumination device for illuminating the surgical field of an electrocautery pencil, the illumination device comprising:
a sleeve with at least one light-emitting component attached to the sleeve.

18. The illumination device according to claim 17, wherein the sleeve defines an open interior, the open interior having a diameter configured to receive the distal end of an electrocautery pencil.

19. The illumination device according to claim 18, wherein the at least one light-emitting component is attached to the sleeve via a flexible wire.

20. The illumination device according to claim 19, wherein the device further comprises a battery attached to the sleeve.

21. An illumination device for illuminating the surgical field of an electrocautery pencil, the illumination device comprising:
a mount having a hole therethrough configured to receive a mounting end of an electrocautery electrode, the mount having at least one light-emitting component attached thereto.

22. The illumination device of claim 21, wherein the at least one light-emitting component is configured to be electrically connected to the electrocautery electrode.

23. The illumination device of claim 22, wherein the mount further comprises a battery connected to the mount and the at least one light-emitting component.
